(19) United States
(12) Patent Application Publication
(10) Pub. No.: US 2008/0119797 A1
(43) Pub. Date: May 22, 2008

(54) System with a syringe device and a needle device

(76) Inventor: Stanley Kim, Upland, CA (US)

Correspondence Address: Stanley Kim, 421 East 25th Street, Upland, CA, 91784, US

(21) Appl. No.: 11/602,101

(22) Filed: Nov. 20, 2006

(51) Int. Cl. A61M 5/34

(52) U.S. Cl. 604/240

(57) ABSTRACT

A system using multiple thin needles instead of a single large needle is less painful when puncturing a body organs because of less tissue injury, and has faster healing time than that of a single large wound, while a sufficient amount of liquids can be delivered efficiently through the multiple lumen of the needles.

A syringe device as well as a combination system of a syringe and a needle are developed to serve the above principle methodologically:
1. A syringe device with multiple connecting tips (FIG. 1).
2. A syringe device comprised of a barrel with multiple divided lumen (FIG. 8), a matching plunger for each said divided lumen (15), and a matching connecting tip in a plurality for each said divided lumen.
3. A combination system in which the above syringe devices (FIG. 1, FIG. 8) are mated with either, or a mixture of, a needle device having a single needle mounted on a single needle hub (FIG. 6), and a needle device having multiple needles mounted on a single common needle hub (FIG. 7).
4. A combination system (FIG. 4) in which a syringe device with a single connecting tip (FIG. 3) is mated with a needle device having multiple needles mounted on a single common needle hub.

### CROSS-REFERENCE TO RELATED APPLICATION

U.S. patent application Ser. 11/420,767

### FEDERALLY SPONSORED RESEARCH

None.

### SEQUENCE LISTINGS

None.

### BACKGROUND

1. Field of Invention

This invention relates to a system with a syringe and hypodermic needles.

Since the advent of a system of syringe and hypodermic needle about 100 years ago, no changes of this basic system have been taken place except the development of disposable system to prevent spread of diseases by using a same needle for many different individuals. We still use a single needle attached or mounted on a single syringe tip.

In a previous invention “U.S. patent application Ser. No. 11/420,767, Stanley Kim et al. introduced a system using multiple thin needles. This system can minimize the tissue injury and pain, while allowing to obtain body fluid or to inject liquid chemicals in a sufficient amount.

In this invention, a new system using a syringe device with multiple connecting tips, and a combination system using a syringe device and a needle device are introduced.

2. Prior Art

Since the invention of a syringe and a hypodermic needle, no changes have been taken place for more than 100 years: A single needle mounted on a single syringe.

Recently, Stanley Kim, et al. have invented a needle device that has a plurality of needle mounted on a body part or a syringe (U.S. patent application Ser. No. 11/420,767).

Neither a syringe device having a plurality of connecting tip, nor a combination system involving a syringe device and a needle device allowing simultaneous puncturing and injecting with multiple thin needles has been known.

### OBJECTS AND ADVANTAGES

As described in the previous invention “U.S. patent application Ser. No. 11/420,767”, the system using multiple thin needles instead of a single large needle is less painful when puncturing skin because of less tissue injury associated with it. In addition, the healing time would be faster with multiple small puncture wounds than that of a single large one.

For example, an ophthalmologist injects a newly developed medicine “Lucentis” into the eyeball directly for the treatment of Macular Degeration. Current method involves the ophthalmologist using a 30 gauge needle to puncture the eyeball and to inject the medicine directly into the eyeball cavity (intravitreous injection). Although a topical anesthesia is used to numb the outer layer of the eyeball, the patient still feels the pain because the inner layers are not be able to be numbed. In addition, the puncture wound in the eyeball can cause bleeding, infection, inflammation, or even leakage of the fluid from the eye cavity.

If the opthalmologist uses a system having two thinner needles with a size of 33 gauge or thinner, instead of a single larger 30 gauge needle, the patient will have less tissue injury, less pain, and faster wound healing. At the same time, the doctor can inject sufficient amount of medicine efficiently through each lumen of the 2 needles. If only a single thin needle is used, it will take much longer to inject the same amount because of the narrower lumen in the thinner needle. In addition, the longer the duration of puncture time, more the injury to the tissues. By using a multiple thin needle system, this problem can be overcome.

One may create various combination of a system serving the above principle:

1. A syringe device with multiple connecting tips mated with a needle device having a single needle mounted on a single needle hub.

2. A syringe device with a single connecting tip mated with a needle device having multiple needles mounted on a single common needle hub.

3. A syringe device with multiple connecting tips in which each syringe tip is mated with a needle device having multiple needles mounted on a single common needle hub. In this system, maximum number of needles can be attached in the syringe device.

4. A syringe device with multiple connecting tips in which each syringe tip is mated with a mixture of a needle device having a single needle mounted on a single needle hub, and a needle device having multiple needles mounted on a single common needle hub.

5. A syringe device having a barrel with multiple divided lumen, a matching plunger for each said divided lumen, and a matching connecting tip in a plurality for each said divided lumen. In this system, multiple different liquid materials can be injected simultaneously or sequentially through the multiple thin needles.

### SUMMARY

A system using multiple thin needles instead of a single large needle is less painful when puncturing skin or other body organs because of less tissue injury associated with it. In addition, the healing time would be faster with multiple small puncture wounds than that of a single large wound. At the same time, one can inject sufficient amount of liquid materials efficiently through each lumen of the multiple needles simultaneously.

A syringe device as well as a combination of system of a syringe and a needle are developed to serve the above principle methodologically:

1. A syringe device with multiple connecting tips.

2. A syringe device comprised of a barrel with multiple divided lumen, a matching plunger for each said divided lumen, and a matching connecting tip in a plurality for each said divided lumen.

3. A combination system in which the above syringe devices are mated with either, or a mixture of, a needle device having a single needle mounted on a single needle hub, and a needle device having multiple needles mounted on a single common needle hub.

4. A combination system in which a syringe device with a single connecting tip is mated with a needle device having multiple needles mounted on a single common needle hub.

### FIGURES

FIG. 1 is a perspective view of a syringe with 2 connecting tips.

FIG. 2 is a perspective view of a syring with 2 connecting tips wherein each connecting tip is mated with a needle device having a single needle mounted on a single needle hub.

FIG. 3 is a perspective view of a syringe with single connecting tip.

FIG. 4 is a perspective view of a syringe with a single tip that is mated with a needle device having 2 needles mounted on a single common needle hub.

FIG. 5 is a perspective view of a syringe with 2 connecting tips that are mated with 2 separate needle devices wherein each needle device has 2 needles mounted on a single common needle hub.

FIG. 6 is a needle device wherein a single needle is mounted on a single needle hub.

FIG. 7 is a needle device wherein 2 needles are mounted on a single common needle hub.

FIG. 8 is a perspective lateral view of a syringe device having 2 small chamber lumen in a common larger barrel, wherein each small chamber lumen has 2 connecting tips.

FIG. 9 is a perspective view of a syringe device having 2 small chamber lumen in a common larger barrel, wherein each small chamber lumen has 2 connecting tips.

### REFERENCE NUMERALS

10. A connecting tip of a syringe
11. A plunger of a syringe
12. A barrel of a syringe
13. A needle hub
14. A needle
15. A small chamber lumen within a common larger barrel
16. A common larger barrel

### DETAILED DESCRIPTION

A syringe has 3 components: A barrel (12), a plunger (11), and a connecting tip (10). The liquid materials filled in the syringe barrel (12) are injected into a target object through the connecting tip (10) by pushing the plunger (11) forward.

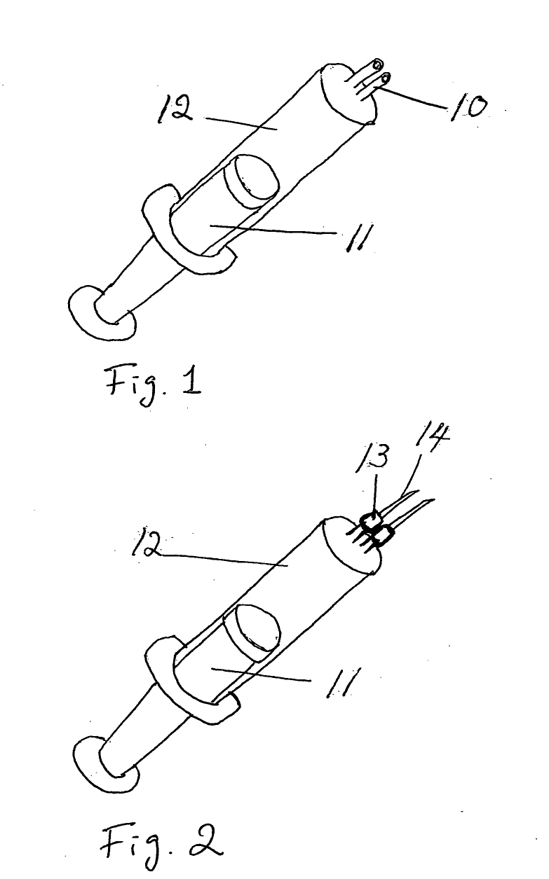

FIG. 1 is a perspective view of a syringe device having 2 connecting tips (10).

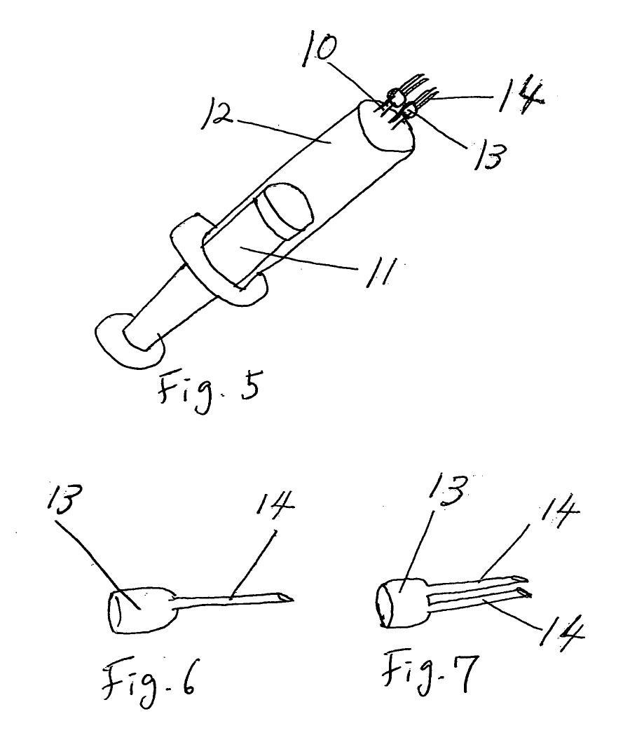

FIG. 2 is a perspective view of a syringe device having 2 connecting tips on which a needle device (FIG. 6) is attached and fit separately. In this needle device, a single needle is mounted on a single needle hub (FIG. 6).

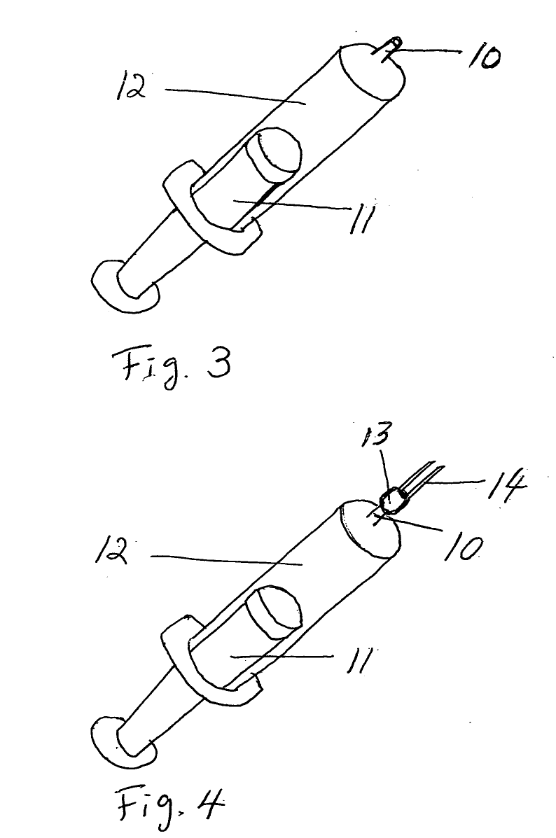

FIG. 3. is a perspective view of a syringe device with a single connecting tip (10). All of the commercially available syringe has a single tip.

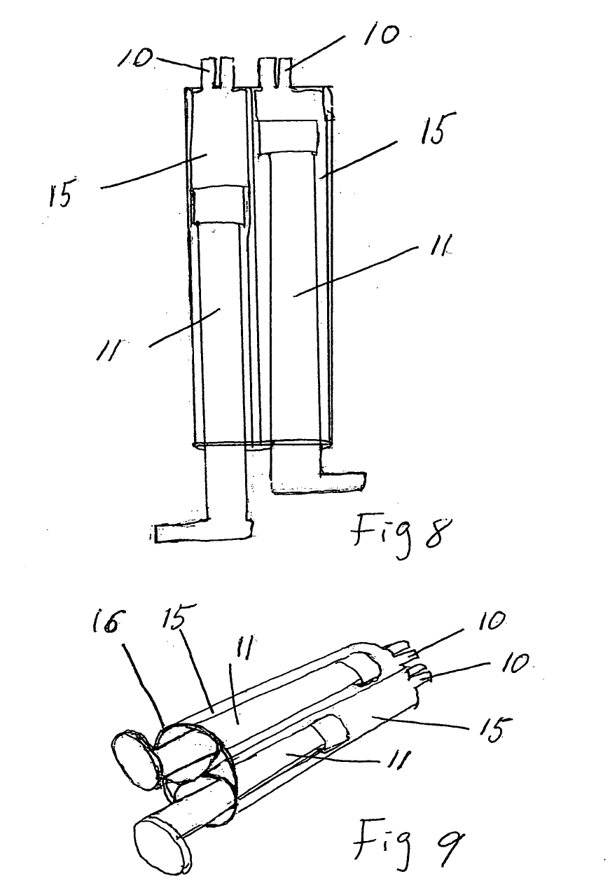

FIG. 4 is a perspective view of a syringe device with a single connecting tip (10) mated with a needle device having 2 needles mounted on a single common needle hub (FIG. 7).

FIG. 5 is a perspective view of a syringe device having 2 connecting tips. Each connecting tip is mated with a needle device (FIG. 7) having 2 needles mounted on a single common needle hub. In this system of FIG. 5, the syringe can have 4 needles attached because each needle device has 2 needles mounted on it.

FIG. 6 is a perspective view of a needle device comprised of a single needle (14) and a needle hub (13).

FIG. 7. Is a perspective view of a needle device comprised of 2 separate needles (14) mounted on a single common needle hub (13).

FIG. 8 is a perspective lateral view of a syringe having 2 small chamber lumen (15) within a large common barrel (16). The each small chamber lumen (15) has its own 2 connecting tips (10).

FIG. 9 is a perspective view of a syringe device wherein a larger common barrel (16) has 2 small chamber lumen (15). The each small chamber lumen has its own 2 connecting tips (10).

Although the description above contained many specificities, these should not be construed as limiting the scope of the invention but as merely providing illustrations of some of the presently preferred embodiments of this invention. Thus the scope of the invention should be determined by the appended claims and their legal equivalents, rather than by the examples given.

### OPERATION

A needle device (FIG. 6) (FIG. 7) is attached and fit in the syringe tip (10). The syringe barrel (12) is filled with liquid materials by transferring the liquids from a container such as a drug vial by way of the pulling the plunger (11) backward. A different large bore needle can be used for the purpose of transferring the liquids from the container.

After filling the barrel (12) with a desirable amount of the liquids, the needle(14) will puncture the targer object, such as skin. Once the needle reaches the target object in an optimum depth, the plunger (11) will be pushed forward to inject the lquids out of the barrel (12), transferring the liquids to the target onject.

Once the desirable amount of the liquids are injected into the target object, the whole system with the syringe and the needles is withdrawn from the target object.

In a syringe system of FIG. 8 and FIG. 9, each small chamber lumen (15) is filled with different kind of liquid materials separately. After each chamber lumen is filled with the liquids, a single poke with the syringe having multiple needles is made. Once the needles reached the target object, the plunger of each chamber barrel is pushed forward to transfer the liquids to the target objects, either simultaneously or sequentially.

I claim:

1. A syringe device comprising of a barrel, a plurality of means for transferring a material in or out of said barrel such as multiple connecting tips, and means for filling or emptying said barrel by creating either positive or negative pressure in said barrel such as a plunger. Said syringe device has various kinds of size, shape, materials, configurations, color, or other features.

2. A syringe system of claim 1, wherein said barrel is comprised of a plurality of smaller chamber lumen within said barrel, each said chamber lumen has a plurality of means for transferring a material in or out of each said chamber lumen separately such as multiple connecting tips, and means for filling or emptying said chamber lumen such as a plunger that are matched with said small chamber lumen in number.

3. A syringe device of claim 1, further including a needle device to be used as a combination system, wherein said needle device is comprised of a plurality of needle or cannula mounted on a single needle hub. Said needle hub is a place where a syringe device is attached to fit in securely. Said needle device has various kinds of size, length, shape, material, configuration, color, or other features. Said needle and said hub can be constructed as a single piece, or as two separate pieces that are attached or bonded together as a one body. Said combination system can be attached as one piece, or can be detachable.

4. A syringe device of claim 2, further including a needle device to be used as a combination system, wherein said needle device is comprised of a plurality of needle or cannula mounted on a single common hub. Said hub is a place where means for transferring materials through, such as a connecting tip of the syringe, is attached to fit in. Said needle device has various kinds of size, length, shape, material, configuration, color, or other features. Said needle and said hub can be constructed as a single piece, or as two separate pieces that are attached or bonded together as a one body. Said combination system can be attached as one piece, or can be detachable.

5. A combination system of claim 3, wherein said needle device is comprised of a single needle or cannula mounted on a single needle hub.

6. A combination system of claim 4, wherein said needle device is comprised of a single needle or cannula mounted on a single needle hub.

7. A syringe device of claim 1, further including a needle device to be used as a combination system, wherein said needle device is comprised of a mixture of a needle device of claim 3 that has a plurality of needle mounted on a needle hub, and a needle device of claim 5 that has a single needle mounted on a needle hub. Said combination system can be attached as one piece, or can be detachable.

8. A combination system of claim 7, wherein said syringe device is comprised of a syringe device of claim 2.

9. A combination system of claim 3, wherein said syringe device has a single connecting tip instead of multiple connecting tips.

10. A combination system of claim 4, wherein said syringe device has a single connecting tip instead of said multiple connecting tips.
